(19) United States
(12) Patent Application Publication
(10) Pub. No.: US 2023/0248229 A1
(43) Pub. Date: Aug. 10, 2023

(54) CATHETER AND CATHETER KIT INCLUDING SUCH CATHETER

(71) Applicant: Creo Medical Limited, Chepstow, Monmouthshire, South Wales (GB)

(72) Inventors: Chris Hancock, Chepstow (GB); George Ullrich, Bethesda Gwynedd (GB); David Webb, Bethesda Gwynedd (GB); David Woods, Chepstow (GB)

(73) Assignee: Creo Medical Limited, Chepstow, Monmouthshire, South Wales (GB)

(21) Appl. No.: 18/015,516

(22) PCT Filed: Jun. 22, 2021
(86) PCT No.: PCT/EP2021/067004

§ 371 Date: Jan. 10, 2023

(30) Foreign Application Priority Data
Aug. 5, 2020 (GB) 2012132.3

(51) Int. Cl. A61B 1/018; A61B 1/00; A61B 1/04

(52) U.S. Cl. CPC A61B 1/018; A61B 1/00121; A61B 1/04

(57) ABSTRACT

A catheter (10) configured to be inserted into a scoping device (12). The catheter comprises a body (30) having a distal end and a proximal end, and an insertion device (28). The body includes at least a first lumen extending between the distal end and the proximal end, the first lumen being configured to receive at least one of a guide wire (24), a camera (52), an ablation device (54) and a probe device (56). The insertion device is connected to the body at the proximal end and includes a first passage for receiving the guide wire and a second passage for receiving one of the camera, the ablation device and the probe device. Some other embodiments provide a catheter kit.

### TECHNICAL FIELD

The invention refers to a catheter configured to be inserted into a scoping device and including a body having a distal end and a proximal end. The invention further relates to a catheter kit comprising such a catheter.

### BACKGROUND

Endoscopes such as duodenoscopes are commonly used in surgeries. Duodenoscopes are commonly used in Endoscopic Retrograde Cholangiopancreatography (ERCP). A commonly used duodenoscope includes a handpiece and an insertion tube which is inserted via the mouth to reach the pancreas and is guided by the handpiece. The duodenoscope includes a biopsy port or work port which is connected to a biopsy channel or work channel extending through the insertion tube. A surgical instrument may be moved out of the distal end of the insertion tube in order to perform various procedures. For each different kind of procedure, a different instrument is needed and, thus, needs to be moved through the work channel or biopsy channel. Because of the length of the work channel, this is cumbersome and time-consuming.

### SUMMARY

The invention is defined by the independent claims. The dependent claims describe preferred embodiments of the invention.

A catheter is configured to be inserted into a scoping device such as an endoscope. The catheter includes a body and an insertion device. The body has a distal end and a proximal end. The body includes at least a first lumen extending between the distal end and the proximal end, wherein the first lumen is configured to receive at least one of a guide wire, a camera, an ablation device and a probe device. The insertion device is connected to the body at the proximal end and includes a first passage for receiving the guide wire and a second passage for receiving one of the camera, the ablation device and a probe device.

A catheter kit comprises a catheter as described above, a guide wire, and one or more of the group including a camera, an ablation device and a probe device.

A method for handling a catheter, wherein the catheter includes a body having a distal end and a proximal end, wherein the body includes at least a first lumen extending between the distal end and the proximal end, the method comprising the steps of inserting a guide wire into the lumen of the body at the proximal end, pushing the guide wire through the lumen until the guide wire protrudes from the distal end, sliding the body over the guide wire until the distal end of the body is located at a distal end of the guide wire, removing the guide wire from the lumen while the body is not moved, inserting one of a camera, an ablation device and a probe device into the body into the lumen of the body at the proximal end, and pushing the one of the camera, the ablation device and the probe device through the lumen until a distal end of one of the camera, the ablation device and the probe device protrudes from the distal end of the body.

The catheter allows to easily position one or more of the camera, the ablation device and the probe device at a point within cavity in the human body in order to perform surgery and/or analyse/probe/examine a particular body part. This is achieved in that the guide wire allows to position the catheter at the desired location. After retraction of the guide wire, the body of the catheter still is positioned at the desired location. For example, the distal end of the body is supported by the tissue of the cavity. After insertion of one or more of the camera, the ablation device and a probe device, these components are correctly located due to the body of the catheter. In addition, the one or more of the camera, the ablation device and a probe device can be easily positioned at the desired location since they solely need to be pushed through lumen of the body. A navigation of these devices within the cavity of the human body is not necessary. Additionally or alternatively, the guide wire can be simultaneously arranged with one or more of the camera, the ablation device and a probe device in the body such that by positioning the catheter using the guide wire, the one or more of the other camera, the ablation device and a probe device are simultaneously positioned.

The provision of the insertion device facilitates the insertion of the guide wire and of the group including the camera, the ablation device and the probe device. The first passage and/or the second passage may be configured/shaped to simplify the insertion into the lumen of the body.

In addition, the first passage and/or the second passage can be configured/shaped such that the insertion of the respective component is simplified. Furthermore, it is possible for the guide wire to remain in the first passage while one or more of the camera, the ablation device and the probe device are arranged in the body of the catheter. If it is necessary to remove the one or more of the camera, the ablation device and a probe device from the body of the catheter, for example from the first lumen, the guide wire does not need to be re-inserted into the catheter since it is still located within the first passage. Similar advantages are present when the one or more of the camera, the ablation device and a probe device are located within the second passage while the guide wire is positioned within the body of the catheter. Thus, the first and second passage allow the components to be maintained within the insertion device, while they are not positioned within the body of the catheter such that they can easily introduced to the body of the catheter.

The catheter is preferably configured to be inserted into an insertion tube of the scoping device, such as an endoscope. A section of the catheter may be guided through a work channel or biopsy channel of the insertion tube. To this end, the body is elongated and has an outer diameter that is smaller than an inner diameter of the work channel or biopsy channel. The scoping device may be a duodenoscope, a bronchoscope, an endoscope for performing brain surgery or any other type of endoscope with which a minimally invasive surgery can be performed.

The guide wire can be a commonly used guide wire to position a catheter at a desired location.

The scoping device may further comprise a handpiece with which the insertion tube can be guided through the cavities within the human body. The handpiece may also provide further commonly known functionalities which are needed for endoscopic procedures.

The camera, the ablation device, and the probe device may be instruments regularly needed for conducting Endoscopic Retrograde Cholangiopancreatography (ERCP). The kit and/or the catheter may also be employed for endoscopic lung surgery and/or endoscopic brain surgery.

The camera may include an optical sensor and/or a light source at its distal end. The optical sensor and the light source may be connected via wires through the allocated body to a display device. Alternatively or additionally, the camera may include an optical fibre with which images captured at a distal end are guided to the proximal end of the camera. The camera may also include optics at its distal end for focusing on the region to be imaged. The camera may include an optical fibre for guiding light from the proximal end to the distal end in order to illuminate the cavity within the human body.

The ablation device can be a commonly known ablation device and may be configured to emit microwave electrical energy and/or radio frequency (RF) electrical energy at its distal end in order to ablate, coagulate and/or cut tissue within the cavity of the human body. The ablation device may be capable of both bipolar radio frequency and microwave energy delivery. The ablation device may have the functionality of an adjustable ablation profile, of thermal management, of a local current path, and/or of a larger ablation.

The ablation device may configured and/or shaped as described in WO 2020/011547 A1 or WO 2020/089015 A1 or WO2017/174513.

The ablation device may include the functionality of performing remote sensing by microwave radiometry. Microwave radiometry is a non-invasive method for sensing temperature in the surroundings of the probe. For example, the antenna of the ablation device may be used to detect the microwave radiometric signals in order to create a radiometric image of the tissue surrounding the probe. Thus, the ablation device may be used to gather images depicting the temperature of the tissue.

The probe device may be any endoscopic device with which tissue within a human body can be analysed and/or examined. The probe device may be a Raman spectroscopy device for performing Raman spectroscopy at the distal end of the Raman spectroscopy device. The Raman spectroscopy device may be a commonly known probe device. The probe device may be capable of conducting a live biopsy by using Raman scattering of light. In particular, the Raman spectroscopy device may be capable of generating images of the tissue defining the cavity within the human body by using Raman scattering.

The Raman spectroscopy device may include several optical fibers extending between the distal end and the proximal end, in particular between the distal end and an analysis device for analyzing the scattered light. One or more of the optical fibers may be configured to guide light from a light source, such as a laser, to the distal end. The laser emits light having a wavelength which is suitable for Raman scattering. Effective wavelength are between 780 nm and 1000 nm, preferably 845 nm.

One or more of the optical fibers are provided for guiding the scattered light collected at the distal end to the analysis device for analyzing the Raman scattering of the tissue at the desired location. The Raman spectroscopy device may include one or more optical lenses for focusing light to the tissue and/or the scattered light into the optical fibers. The optical lens may be a ball lens. Furthermore, optical filters, such an excitation filter and/or in collection filter, may be arranged at the distal end of the Raman spectroscopy device. The analysis device may include a spectrometer for analyzing the intensity of the scattered light depending on the wavelength. The spectrometer may include a spectrograph and a detector.

The Raman spectroscopy device and/or the Raman spectroscopy image may be used for taking a “live biopsy”; the Raman spectroscopy device may be used for determining whether the tissue at the desired location is cancerous or not.

The Raman spectroscopy device may include a camera which can be constituted by at least two additional optical fibers one of which is for guiding (visible) light from a light source (for example emitting light having a wavelength between 635 nm and 700 nm) to the distal end and one of which is for guiding light gathered at the distal end to a video processor. The camera built in to the Raman spectroscopy device may be used to determine where the Raman spectroscopy device is taken. Preferably, the wavelength of the light for the camera and the light for the Raman spectroscopy device do not overlap such that no interference occurs between the light for the Raman spectroscopy device and the light for the camera.

The probe device may include devices for optical coherence tomography (OCT) and/or devices for gathering fluorescence signals in the cavity of the human body. For example, fluorescent dyes may be flushed into the cavity and the fluorescent light is gathered by the probe device. The probe device may also be configured to provide the light for exciting the fluorescent dyes.

The elongated body of the camera, the ablation device and/or probe device may be flexible while a distal end region may be stiff in order to provide the sensors and/or other components necessary for performing the respective function. In case of a stiff distal end region, the stiff distal end region may be short enough that it can be guided through the catheter and the insertion tube.

The body of the catheter may be flexible such that it can be guided or arranged within the insertion tube of the scoping device during manoeuvring the insertion tube through the human body. The distal end of the body is configured to be arranged at the distal end of the insertion tube and can be pushed out of the distal end of the insertion tube. The proximal end of the body is intended to protrude from a biopsy port or a work port of the scoping device. Thus, the body has a length which is longer than the length from the biopsy port to the distal end of the insertion tube. The biopsy port or work port may also be considered a device entry point.

The first lumen is open at both the distal end and the proximal end. The body may include an outer shell and an inner material structure which provides the first lumen. The outer shell and the inner material structure may be made from the same or different flexible materials. The outer shell may be made from material which protects the body from the bodily fluids at the distal end of the insertion tube. The outer shell may be heat shrunk onto the inner material structure. The inner material structure may be made from polyamide (PA), Polytetrafluoroethylene (PTFE) and/or Polybutylacrylate (PBAK).

The first lumen may have a diameter which is slightly greater than the diameter of the elongated body of the camera, the ablation device and the probe device such that a clearance is provided between the elongated body and the surface of the first lumen.

The first lumen may be provided solely by the inner material structure. Alternatively, the outer shell as well as the inner material structure may define the first lumen. The inner material structure may provide the stability to the body and, thus, span the first lumen. The inner material structure may include an inner passage which constitutes the first lumen. The inner material may additionally or alternatively include a channel open to the surrounding which is covered by the outer shell such that the first lumen is separated from the surrounding of the body.

The surface of the inner material structure defining the first lumen may be provided with a lubricant which may be inserted into the first lumen and/or the inner material structure stores the lubricant. The inner material structure may be manufactured by extrusion. The first lumen may be provided with a liner tube which may help to reduce the friction between one of the camera, the ablation device and the probe device and the inner material structure. The liner tubes may be made from polyamide (PA) and/or Polytetrafluoroethylene (PTFE).

The first lumen may only have an opening at the distal end and the proximal end such that the first lumen continuously extends between the first end and the second end. The body preferably provides the first lumen.

The insertion device is connected to the body at the proximal end in order to simplify the insertion of the guide wire, the camera, the ablation device and/or the probe device. To this end, the insertion device is in (fluid) communication with the first lumen. The first passage and the second passage may be each connected to the first lumen. Alternatively, the first passage and the second passage may be connected to a common passage which is connected to the body. The first passage and the second passage each have a certain length such that is possible to retain the guide wire on the one hand, and one of the camera, the ablation device and the probe device on the other hand, respectively.

The liner tube may extend beyond the proximal end of the body and, in particular, into the insertion device, for example into the common passage, the first passage and/or the second passage. The liner tube may be permanently fixed to the common passage, the first passage and/or the second passage. This simplifies the connection of the insertion device to the first lumen such that there is a (fluid) communication between the insertion device and the first lumen.

The first passage and/or the common passage may extend in a longitudinal direction which can be understood as the extension of the body at the proximal end. The longitudinal direction may also be defined by the extension of the insertion device. The second passage may branch off the common passage such that the second passage may be inclined to the longitudinal direction. The first passage may extend parallel to the longitudinal direction and/or coaxial to the first lumen or the common passage.

The first passage may include a first port which facilitates the insertion of the guide wire into the first passage and, therefore, into the catheter. The guide wire port may include a funnel-shaped structure or funnel.

The second passage may include a second port which may also simplify the insertion of one or more of the camera, the ablation device and a probe device. The second port may also contribute to holding one or more of the camera, the ablation device and a probe device at a certain position. To this end, the second port may include an outer surface having a higher friction compared to the second passage. Alternatively or additionally, the second port may include means for locking one or more of the camera, the ablation device and a probe device at a certain position. The first port and/or the second port may be removable from the first passage and the second passage, respectively.

The second passage and the first lumen have an inner diameter which is slightly larger than an outer diameter of the elongated body of the camera, the ablation device and a probe device. The inner diameter of the first passage may be smaller than the inner diameter of the second passage and the first lumen since the outer diameter of the guide wire is usually smaller than the outer diameter of the elongated body of the camera, the ablation device and a probe device.

According to an optional embodiment, the camera, the ablation device, and/or the probe device include a bulge and an elongated body. The elongated body may include a first section and a second section which can be visually differentiated from the first section and is positioned between the first section and the bulge. Optionally, a length of the first section is the same as a length of the body. Further optionally, the first section is more flexible than the second section.

The bulge may be a connector for connecting the camera to a display device, the ablation device to a generator and/or the probe device to analysis device which may include a computer and a display. The bulge has an outer diameter which is greater than the outer diameter of the elongated body and, in particular greater than the inner diameter of the second passage. Thus, the bulge may act as a stopper for preventing that the camera, the ablation device and/or the probe device are pushed to far into the body of the catheter.

Preferably, the length of the elongated body corresponds to the length of the catheter. In particular, the length of the first section corresponds to the sum of the lengths of the body, the common passage and/or the second passage. The length of the second section may be chosen with regard to the desired length with which the camera, the ablation device and/or the probe device can be moved out of the distal end of the catheter.

The first section and the second section may have outer surfaces that are made from different materials. Alternatively and/or additionally, the outer surfaces of the first section and a second section may have different colours and/or different surface structures such that they can be visually differentiated from each other. Alternatively or additionally, the second section may include markers, labels and/or scales which indicate how much the elongated body is inserted into the catheter. The markers, labels and/or scales may be printed onto the second section and/or may be protrusions arranged on the second section. The markers, labels and/or scales can be used to determine how far the elongated body of the camera, the ablation device and the probe device is pushed out of the distal end of the body.

The second section may be less flexible than the first section. The second section can be rigid, for example by providing a rigid sleeve over the elongated body at the position of the second section. The rigidity of the second section may facilitate that the elongated body does not bend when protruding from the insertion device. This may reduce swinging of the elongated body and, thus, facilitates that the elongated body and/or the bulge may be easily gripped. However, the second section is flexible enough to push in from the second passage to the common passage which can be inclined to the second passage.

The guide wire may have an outer diameter which does not change along its extension, i.e. a constant outer diameter.

According to an optional embodiment, the insertion device is rigid.

The common passage, the first passage, and/or the second passage may be rigid. The rigidity of the insertion device may help to ensure that the guide wire, the camera, the ablation device and/or the probe device do not bend after exiting the body of the catheter. This may reduce swinging of the elongated bodies and/or the guide wire such that these devices may be more easily gripped. Furthermore, the rigidity of the insertion device may help to support the weight of the camera, the ablation device and/or the probe device. In particular, the elongated body of the ablation device may be heavy such that the insertion device provides an additional support. This means the rigidity of the insertion device is chosen such that it can support the weight of the guide wire, the camera, the ablation device and/or the probe device.

According to an optional embodiment, the insertion device further includes a third passage for introducing a fluid into the body and a fourth passage for emitting the fluid from the body.

The third passage may include a third port which may be configured as a connector to a fluid line. The fourth passage may include a fourth port which may be configured as a connector to a fluid line. The third passage be used for introducing fluid, such as water, into the body of the catheter which then flows out of the catheter that the distal end. The third passage may be connected to a fluid source which introduces the fluid with pressure into the catheter.

The fluid may be used for flushing/irrigating the cavity within the human body and/or for rinsing the distal end of the camera. Alternatively or additionally, the markers and/or (fluorescent) dyes may be flushed to the cavity which facilitate the capture of images of the cavity such as fluorescence images.

The fourth passage may be connected to a suction source which generates low pressure for sucking fluid out of the catheter and, thus, out of the cavity within the human body.

According to an optional embodiment, the catheter further comprises a support device configured to be connected to the biopsy port or work port of the scoping device, wherein preferably the support device can be rotatably connected to the biopsy port.

The support device may be made from a rigid material and serves to provide support for the body and/or the insertion device with respect to the scoping device. The support device may have an elongated shape whose axis of extension is parallel or coincides with the longitudinal direction of the first passage.

The support device may be attached to the biopsy port of the scoping device by means of a leur lock. Alternatively, the support device may include a fixing portion for fixedly attaching the support device to the biopsy port while the fixing portion may be rotatable with respect to the rest of the support device.

The provision of rotation of the support device with respect to the scoping device allows to rotate the support device and, thus, the insertion device to a desired orientation which helps to provide easy access to the first passage and/or the second passage. The support device may be locked to the biopsy port of the scoping device in a fluid tight manner.

According to an optional embodiment, the support device includes a support body configured to be connected to the biopsy port and a slider slidable with respect to the support body, when the proximal end of the body is fixed to the slider.

The support body may be made from a rigid material and may be rotatable with respect to the scoping device. The support body may have an elongated shape. The support body may be fixed with respect to the scoping device and, thus, the insertion tube.

The slider may slide in the longitudinal direction, e.g. along the elongated shape of the support body. Thus, the slider may allow movement of the body into the insertion tube and/or out of the insertion tube. The functionality of the slider may be therefore regarded as providing a means for moving the body in into and out of the insertion tube of the scoping device.

The slider may be connected to the proximal end of the body or to the common passage. The slider may slide along the support body. Preferably, the support body includes a channel extending in the longitudinal direction wherein the slider slides within this channel. The support body may include a sliding means which facilitates that the slider can freely move with respect to the support body in the longitudinal direction.

The slider may be used for moving the body out of the distal end of the insertion tube. Thus, the slider may be used after the guide wire has been successfully positioned at the desired location. Then, the slider is moved with respect to the support body in order to slide the body of the catheter over the guide wire to position the distal end of the body of the catheter at the desired location.

According to an optional embodiment, the support device further includes a locking device for releasably fixing the slider to the support body.

The locking device is any device that can be used to releasable fix the slider to the support body. In one embodiment, the locking device is a screw which can be screwed into a thread in the slider. The support body may include an elongated slit extending along the channel wherein the screw extends through the slit. By tightening the screw, the slider is pressed against the part of the support body which defines the channel whereby the friction between the slider and the support body fixes the slider to the support body. Loosening the screw, provides a gap between the slider and a channel such that the slider can be moved along the channel while the screw moves within the elongated slit. The screw and the slit can be considered a sliding means.

According to an optional embodiment, the support device includes a locking means for releasably locking the guide wire and/or an orientating device for slidably supporting the guide wire in the longitudinal direction of the support device.

The locking structure is preferably provided for releasably fixing the guide wire with respect to the support body. The locking means is preferably arranged on the support body. The locking means allows to releasably lock movement of the guide wire in the longitudinal direction. In particular, due to the arrangement of the locking means at the support device, the locking means releasably fixes the guide wire such that it cannot move with regard to the support device. By locking the guide wire, the body of the catheter can be slid over the guide wire, for example by using the slider.

The locking means can have any configuration which allows to releasably lock the guide wire with respect to the support device. For example, the guide wire may be clamped. Thereto, the locking means includes a flap which can be folded with regard to the support device. The flap may include one opening, for example an elongated opening, which allows to clamp the guide wire by folding the flap onto or away from the support device.

The orientating device may support the guide wire after the guide wire exits the first passage. In particular, the orientating device supports the orientation of the guide wire in the longitudinal direction. This helps to support the guide wire after exit of the first passage such that the guide wire does not interfere with the camera, the ablation device and/or the probe device. Furthermore, the orientating device may simplify the insertion of the guide wire. For example, the guide wire may first be introduced into the orientating device and, due to the support of the orientating device, the guide wire can be easily inserted into the first passage.

The orientating device may include a central opening and a labyrinth shaped slit which provides a passage between the central opening and the surrounding of the support device. The labyrinth shaped slit allows a sideway introduction of the guide wire into the central opening while ensuring that the guide wire remains in the central opening. The central opening may be an elongated channel which provides support for the guide wire such that the guide wire remains orientated in the longitudinal direction. The extension of the central opening may be parallel to the central opening and, in particular, coaxial to the first passage. The central opening may be arranged on a side face of the channel opposing the face on which the body of the catheter enters the channel.

According to an optional embodiment, the first passage, the second passage, the third passage and/or the fourth passage are in fluid communication with the first lumen.

This means the guide wire, the camera, the ablation device, the probe device and/or the fluid introduced into the body of the catheter are all within the first lumen. In this embodiment, the inner diameter of the first lumen is chosen to be slightly greater than an outer diameter of one of the camera, the ablation device and the probe device. Thus, it is solely possible that one of the guide wire, the camera, the ablation device and the probe device is arranged within the first lumen at a time.

For example, one embodiment of handling the catheter is described in the following. Optionally, at the beginning, the guide wire is introduced into the first passage and the camera is introduced into the second passage but they do not enter the common passage or the first lumen. For manoeuvring the body of the catheter to the desired location, the guide wire is inserted into the first lumen and out of the distal end of the body of the catheter. If a distal end of the guide wire is at the desired location, a position of the guide wire relative to the body and, thus, to the support body is fixed using the locking device. As the slider moves relative to the support body, the slider may be used to slide the body over the guide wire such that the distal end of the guide wire and a distal end of the body of the catheter coincide. Then, the guide wire is retracted from the first lumen but the distal end of the guide wire may still be positioned within the first passage.

After that, one of the camera, the ablation device and the probe device may be moved through the first lumen such that their distal end coincides with the distal end of the body of the catheter. The insertion of the camera, the ablation device or the probe device is simplified since these components can be inserted into the insertion device prior to the insertion into the first lumen. If the ablation device is needed in a cavity within the human body and the camera is arranged within the first lumen, the camera may be removed from the first lumen and the first passage and the ablation device is inserted. As the distal end of the body remains at the desired location while swapping the ablation device and the camera, the positioning of the distal end of the ablation device at the desired location is achieved by pushing the elongated body of the ablation device through the first lumen. This can be repeated with the probe device.

According to an alternative embodiment, the body further includes a second lumen extending between the distal end and the proximal end, wherein preferably the first passage is in communication with the first lumen and the second passage is in communication with the second lumen.

The second lumen is separated from the first lumen by the body. Preferably, the second lumen has only openings at the proximal end and a distal end. The second lumen may be considered as a continuous passage, tunnel or tube extending between the proximal end and distal end. The description of the first lumen may equally apply to the second lumen except when a difference is explicitly stated.

The second passage may be directly attached to the second lumen while the first passage may be directly attached to the first lumen. Alternatively, the common passage may be attached to the proximal end of the body while the first passage and the second passage extend within the common passage or are attached to common passage. For example, the common passage is divided into two passages or tubes which are respectively connected to the first passage and the second passage.

Optionally, the first lumen and the second lumen each include a liner tube. The liner tube of the first lumen may extend into the first passage and/or the liner tube of the second lumen may extend into the second passage. In this way, a simple connection of the first and second passage with the respective lumen can be achieved.

A mode of handling the catheter of this embodiment may start similar to the mode of handling of the previous embodiment. The guide wire is pushed out of the distal end of the body of the catheter in order to position the distal end of the guide wire at the desired location. As the guide wire is arranged within the first lumen and one of the camera, the ablation device and the probe device is arranged within the second lumen, moving the slider to slide the body of the catheter over the guide wire positions the distal end of one of the camera, the ablation device and the probe device at the desired location. It is noted that the body is in a fixed relationship to the slider and, thus, to the insertion device. Moving the body simultaneously moves the device arranged within the second lumen.

Similar to the previous embodiment, one of the camera, the ablation and the probe device may be swapped with one of the two others by removing the no longer required component from the second lumen and the second passage and inserting one of the two others into the second passage and, therefore, into the second lumen.

According to an optional embodiment, the body further includes a third lumen extending between a distal end and a proximal end and/or a fourth lumen extending between distal end and a proximal end, wherein preferably the third passage is in fluid communication with the third lumen and/or the fourth passage is in fluid communication with the fourth lumen.

The third lumen may be used for introducing fluid into the body cavity while the second lumen may be used for sucking the fluid out of the cavity within the human. Here again, the third passage and/or the fourth passage may be directly connected to the third lumen and/or the fourth lumen, respectively. Alternatively, the common passage may be attached to the proximal end of the body while the third passage and/or the fourth passage extend within the common passage or are attached to common passage. For example, the common passage includes one or two additional passages or tubes which are respectively connected to the third passage and/or the fourth passage.

According to an alternative embodiment, the body further includes a fifth lumen extending between a distal end and a proximal end, wherein preferably the insertion device includes a fifth passage and wherein further preferably the fifth passage is in communication with the fifth lumen.

The fifth passage may be directly connected to the fifth lumen. Alternatively, the common passage may be attached to the proximal end of the body while the fifth passage extends within the common passage or is attached to common passage. For example, the common passage includes one additional passage or tube which is connected to the fifth passage. The fifth passage may also branch off from the common passage, similar to the second passage. The fifth passage may also be made from a flexible material. The second passage and the fifth passage may branch off at the same location in the longitudinal direction. The second passage and the fifth passage may also have the same inclination to the longitudinal direction. The fifth passage may also include a fifth port which may be configured or shaped similar or identical to the second port.

According to an optional embodiment, the fifth lumen is configured to receive the camera and the second lumen is configured to receive the ablation device or the probe device.

The elongated body of the camera often has a small diameter compared to the diameter of the elongated body of the ablation device and the probe device. What is more, the ablation device and the probe device often include an elongated body having a similar outer diameter. Therefore, the fifth lumen may have an inner diameter smaller than the inner diameter of the second lumen. The second lumen may be used for advancing the ablation device or the probe device while the fifth lumen may be used to advance the camera. The provision of the second lumen in the fifth lumen allows to simultaneously use the camera and one of the ablation device and the probe device. Thus, the camera may be used to monitor the ablation device or the probe device.

The camera, in particular the elongated body of the camera, may be permanently fixed within the fifth lumen. In this case, the distal end of the elongated body of the camera may be flush with the distal end of the body.

The description of the first lumen and/or the second lumen may equally apply to the fifth lumen except where a difference is explicitly stated. Optionally, the fifth lumen includes a liner tube. The liner tube of the fifth lumen may extend into the fifth passage.

A method of handling this embodiment is similar to the ones described above except that the camera and one of the ablation device and the probe device can be simultaneously used.

According to an optional embodiment, the catheter further comprises a radio marker arranged at the distal end of the body.

The radio marker is preferably made from a material which is visible in images taken using an electromagnetic wave in the X-ray frequency range. For example, the radio marker may be used for x-ray images and/or computed tomography (CT) images. The radio marker may enhance the recognition of the distal end of the body in ultrasound images. The absorption of waves used for imaging (such as electromagnetic waves in the X-ray frequency range) of the material of the radio marker is (significantly) higher than the absorption of the surrounding tissue, of other parts of the scoping device and/or of the catheter.

The radio marker is located close to the distal end of the catheter such that the position of the distal end of the catheter can be checked by imaging the radio marker.

The radio marker can be arranged within the body and/or on the outer surface of the body. Alternatively or additionally, the outer surface of the body may include recesses in which the radio marker is arranged. The radio marker is preferably permanently fixed to the body. The radio marker may be one unitary component or is made up of two or more separate components. For example, the radio marker is constituted by one or more rings extending in the circumferential direction of the body. For example, three such rings are provided.

According to optional embodiment, the catheter further comprises a first electrode and a second electrode located on an outer surface of the body at the distal end.

The electrodes can be used for cutting tissue using radio frequency. In this embodiment, the catheter has a further functionality of cutting tissue, namely by means of the electrodes. The first electrode and the second electrode are positioned close to each other such that a radiofrequency electrical field extends between the electrodes to provide the cutting capabilities.

The first electrode and the second electrode are preferably arranged directly or at least close to the distal end of the body. This allows that the cutting capabilities are close to the location where the camera, the ablation device and the probe device can be placed. The first electrode and the second electrode are made from the conductive material, such as metal. The outer surface of the body may be made from nonconductive material, such as plastic, such that the outer surface of the body provides electrical insulation between the electrodes.

According to an optional embodiment, the body includes a sixth lumen extending between the distal end and the proximal end and a seventh lumen extending between the distal end and the proximal end, wherein preferably a first wire is arranged in the sixth lumen and connected to the first electrode and a second wire is arranged in the seventh lumen and connected to the second electrode.

Unlike the first to fifth lumens, the sixth lumen and/or the seventh lumen may not be open at the distal end. The first wire and a second wire arranged in the sixth lumen and the seventh lumen, respectively, may be connected to the first electrode and the second electrode, respectively, before the distal end, for example channels extending in the radial direction connect the first electrode and the second electrode with the sixth lumen and the seventh lumen, respectively. Thus, the first wire and/or the second wire do not completely extent to the distal end but terminate a short distance before the distal end. As such, it may be necessary to close the sixth lumen and/or the seventh lumen the distal end such that no fluid may enter the sixth lumen and seventh lumen.

The inner material structure may be made from a nonconductive material such that the wires are insulated by each other by the inner material structure material. The wires thus maybe not provided with a separate insulation. The diameter of the sixth lumen and/or the seventh lumen is slightly larger than the diameter of the first wire and a second wire, respectively. However, the diameter of the first and second wires is generally smaller than the diameter of the camera, the ablation device and a probe device such that the diameter of the sixth and seventh lumen is smaller than the diameter of the second and fifth lumens.

The first wire and the second wire may extend beyond the proximal end in order to be connected to a generator which provides radiofrequency electromagnetic energy to be supplied to the first electrode and the second electrode. The first wire and the second wire may be permanently fixed to the body and, in particular, to the first electrode and to the second electrode, respectively.

According to an optional embodiment, the first electrode and/or a second electrode have a spiral shape.

As discussed, the first electrode and the second electrode extend on the outer surface of the body. The first electrode and a second electrode having spiral shapes may be interleaved while they are spaced from each other in order to avoid short circuits. For example, when viewed along the longitudinal direction of the body, the arrangement of the electrodes may be: a part of the first electrode, a part of the second electrode, a part of the first electrode, a part of the second electrode et cetera.

Alternatively, the first electrode and the second electrode have different shapes, for example the shape of a ring whereby the two rings corresponding to the first electrode and the second electrode are spaced apart from each other in the longitudinal direction of the body. It is also possible that the first electrode and the second electrode are plate-shaped.

According to an optional embodiment, the first lumen, the second lumen, the fifth lumen, the sixth lumen and/or the seventh lumen have a circular cross-section. This corresponds to that the elongated body of the camera, the ablation device and a probe device and/or the first wire and the second wire may have a circular cross-section. The cross-sectional shape of the third lumen and/or the fourth lumen may be non-circular. For example, the cross-sectional shape of the third lumen and/or the fourth lumen may be chosen in that it best fills up the area of the cross section of the body. When designing the body, one or more of the first lumen, the second lumen, the fifth lumen, the sixth lumen and/or the seventh lumen are provided wherein the third lumen and/or the fourth lumen are provided in the remaining space and the cross-sectional shape of the third lumen and fourth lumen is chosen accordingly.

According to an optional embodiment, the first lumen, the second lumen, and/or the fifth lumen may also be used for flushing, i.e. a fluid such as water may be introduced into the cavity of the human body or sucked out of this cavity by means of these lumens. In particular, the flushing capability is used after removing the guide wire, the camera, the ablation device and/or a probe device from the respective lumens. In this case, the first lumen, the second lumen and/or the fifth lumen are empty and fluid may be flowing there through. To this end, a fluid pressure line and/or a fluid suction line may be connected to the first passage, the second passage and/or the fifth passage, respectively. It may be possible to replace the first port, the second port and/or the fifth port with connectors for connecting the respective passages to the fluid pressure line and/or the fluid suction line.

In this embodiment, the third passage and/or fourth passage may be in fluid communication with one or more of the first lumen, the second lumen and the fifth lumen. In order to avoid that fluid enters the first passage, the second passage and/or the fifth passage, one or more seals may be provided at the entrance to the first passage, the second passage and/or the fifth passage. The seals provide a fluid tight sealing between the guide wire and the first passage and/or the elongated body and the second passage and/or the fifth passage. Thus, fluid in the common passage and/or in one of the lumens may not spill out of the catheter through the first passage, the second passage and/or the fifth passage.

### BRIEF DESCRIPTION OF FIGURES

Embodiments of the invention will be discussed in conjunction with the accompanying drawings. Therein,

FIG. 1a shows a catheter according to a first embodiment attached to a scoping device;

FIG. 1b shows a guide wire of the catheter of FIG. 1a protruding from a body of the catheter;

FIG. 1c shows the guide wire of FIG. 1b protruding from an insertion tube of the scoping device of FIG. 1a;

FIGS. 2a and 2b show the catheter as depicted in FIGS. 1a and 1b, respectively, wherein the guide wire is replaced by a camera;

FIGS. 3a and 3b show the catheter as depicted in FIGS. 1a and 1b, respectively, wherein the guide wire is replaced by a probe device;

FIGS. 4a and 4b show the catheter as depicted in FIGS. 1a and 1b, respectively, wherein the guide wire is replaced by an ablation device;

FIG. 5 shows a support device of the catheter of FIG. 1;

FIG. 6 shows a catheter kit in line with FIGS. 1 to 5;

FIG. 7 shows cross-sectional views of catheters according to the first embodiment having different sizes;

FIGS. 8a to 8c shows a catheter according to a second embodiment with different instruments arranged in the body of the catheter;

FIG. 9 shows the catheter according to the second embodiment attached to the scoping device;

FIG. 10 shows cross-sectional views of the catheter according to the second embodiment having different sizes;

FIGS. 11a to 11f show sketches how to handle the catheter according to the second embodiment;

FIG. 12 shows a distal end of the body of the catheter according to the second embodiment;

FIGS. 13a and 13b show a catheter according to a third embodiment with different instruments arranged in the body of the catheter;

FIG. 14 shows the catheter according to the third embodiment attached to the scoping device;

FIG. 15 shows cross-sectional views of the catheter according to the third embodiment having different sizes;

FIGS. 16a to 16f shows sketches how to handle the catheter according to the third embodiment; and

FIG. 17 shows a distal end of a body of a catheter according to a fourth embodiment.

### DESCRIPTION OF SOME EMBODIMENTS OF THE INVENTION

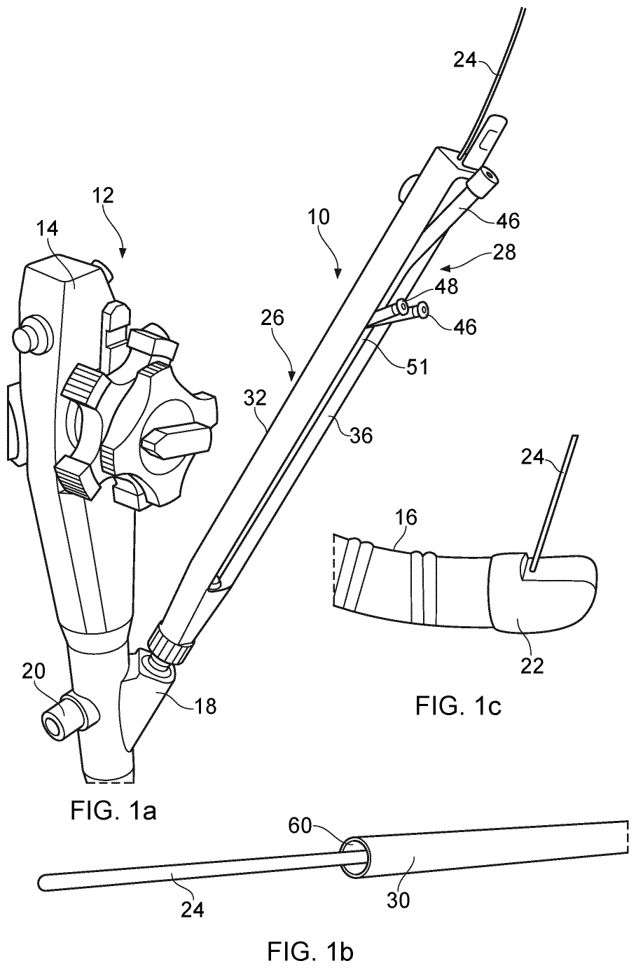

FIG. 1 shows a first embodiment of a catheter 10 attached to a scoping device 12, such as an endoscope which can be a duodenoscope commonly used in Endoscopic Retrograde Cholangiopancreatography (ERCP). The catheter 10 and the scoping device 12 may also be employed in endoscopic lung surgery or endoscopic brain surgery

The scoping device 12 includes a handpiece 14, an insertion tube 16 (better visible in FIG. 1c), a biopsy port 18 and/or a further access port 20. The handpiece 14 may include levers, buttons and/or other actuation means for controlling the scoping device 12. In particular, a head 22 located at the distal end of the insertion tube 16 may be controlled by using the handpiece 14. The head 22 may include mechanisms and/or components common to duodenoscopes such as means for opening and closing an opening within the head 22 such that the catheter 10 may exit the head 22 through the opening.

The insertion tube 16 may include one or more channels which are not visible in the figures. For example, the insertion tube 16 may include a work channel which is connected to the biopsy port 18 (can also be called work port). Another channel may be connected to the access port 20. These channels may be used for advancing instruments and/or fluids to one or more openings in the head 22. In particular, the catheter 10 may be inserted into the work channel via the biopsy port 18. What is more, the catheter 10 may exit the scoping device 12 at the head 22 (as depicted in FIG. 1c; there, only a guide wire 24 of the catheter 12 is depicted) in order to treat, examine and/or analyse a cavity within a human body.

The catheter 10 includes a support device 26, an insertion device 28 and a body 30 having a distal end and a proximal end. The insertion device 28 is attached to the proximal end of the body 30. The insertion device 28 and the body 30 may be attached to the scoping device 12 by means of the support device 26. In particular, the catheter 10 is attached to the biopsy port 18 by means of the support device 26. Preferably, the support device 26 is releasably attachable to the scoping device 12. Furthermore, the support device 26 may be rotatably attached to the biopsy port 18 such that the support device 26 can rotate around an axis defining a longitudinal direction which coincides with the elongated structure of the support device 26.

The support device 26 includes a support body 32 and a slider 34 which can slide relative to the support body 32 in the longitudinal direction of the support device 26. The slider 34 is fixedly attached to the insertion device 28 such that the insertion device 28 can be slid in the longitudinal direction by means of the slider 34.

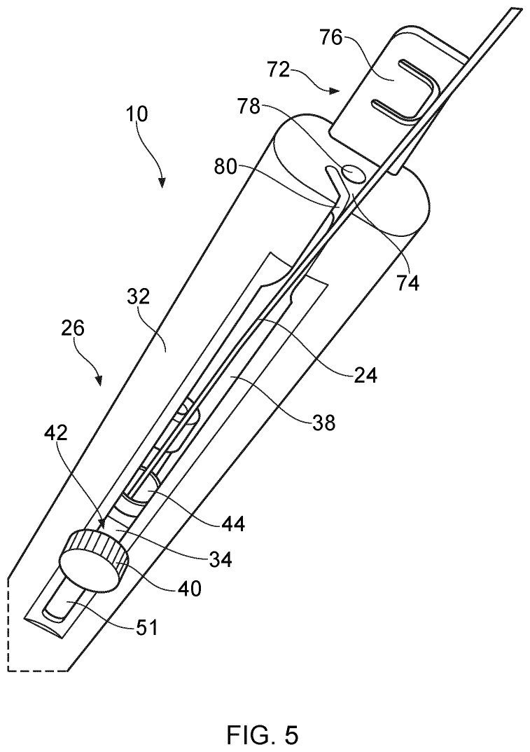

The support body 32 may include a channel 36 in which the slider 34 can slide. One side of the channel 36 is open and another side of the channel 36 may include a slit 38 (see FIG. 5). A screw 40 may extend through the slit 38 and can be screwed into a thread in the slider 34. By tightening the screw 40 into the slider 34, the slider 34 may be releasably fixed to the support body 32. The slit 38 extends parallel to the longitudinal direction. At the same time, the slit 38 and the screw 40 provide a slidable attachment of the slider 34 to the support body 32. The assembly including the slider 34, the slit 38 and the screw 40 may be regarded a locking device 42.

The insertion device 28 includes a first passage 44 (see FIG. 5) and a second passage 46. Optionally, the insertion device 28 further includes a third passage 48 and a fourth passage 50. The insertion device 28 may be made from a rigid material, in particular a rigid plastic material.

The first passage 44, the second passage 46, the third passage 48 and/or the fourth passage 50 may terminate in a common passage 51 which can be connected to the body 30 of the catheter 10. Alternatively, the first passage 44, the second passage 46, the third passage 48 and/or the fourth passage 50 may be directly connected to the body 30. The common passage 51 may also be made from a rigid material and can be fixedly attached to the slider 34.

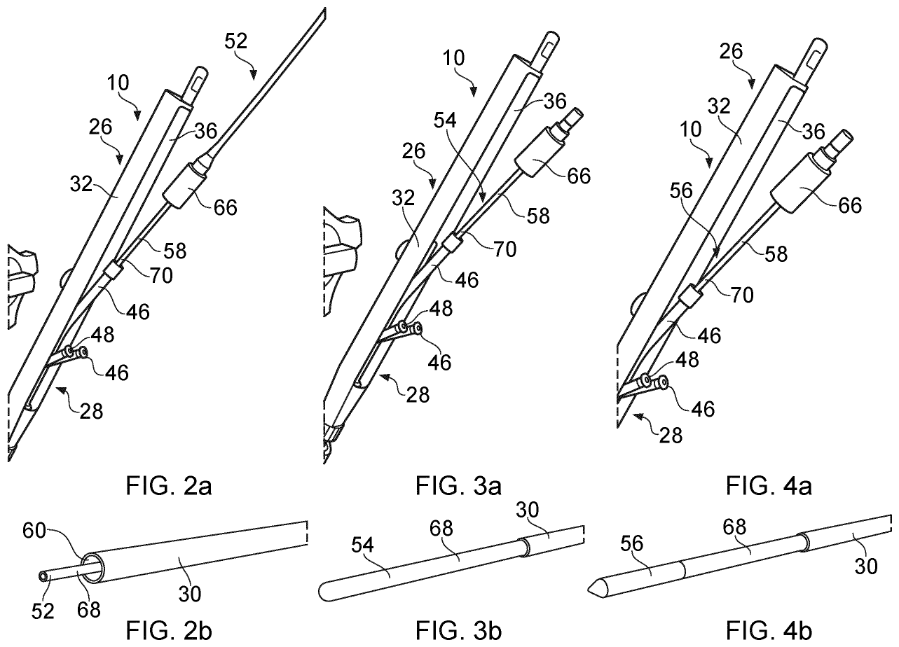

The first passage 44 is provided for facilitating the insertion of the guide wire 24 into the body 30. The first passage 44 may include a funnel at its entrance port (see FIG. 5) which simplifies the insertion of the guide wire 24. The second passage 46 is provided for facilitating the insertion of a camera 52 (see FIG. 2a), an ablation device 54 (see FIG. 2b), and/or a probe device 56 (see FIG. 2c) into the body 30. In particular, an elongated body 58 of the camera 52, the ablation device 54 and/or the probe device 56 is inserted into the body 30.

The body 30 includes a first lumen 60 which extends between the proximal end and the distal end of the body 30. The first lumen 60 is open at the proximal end and at the distal end of the body 30. As better visible in FIG. 7, the body 30 includes an outer shell 62 and an inner material structure 64. The inner material structure 64 defines the first lumen 60, i.e. the first lumen 60 is a hole which extends through the inner material structure 64 from the distal end to the proximal end.

The outer shell 62 may be heat-shrunk onto the inner material structure 64 which can be manufactured by extrusion. The body 30 is flexible such that both the outer shell 62 and the inner material structure 64 are made from flexible materials. An inner surface of the inner material structure 64 defining the first lumen 60 may be lubricated for reducing the friction between the elongated body 58 and the inner material structure 64.

The outer diameter of the body 30 and the inner diameter of the inner material structure 64 (corresponding to the diameter of the first lumen 60) may have the dimensions in millimetre as depicted in FIG. 7. The different diameters allow to house elongated bodies 58 having different outer diameters. The outer diameter of the elongated body 58 is slightly smaller than the inner diameter of the inner material structure 64 allowing for a clearance gap. The outer diameter of the elongated body 58 of the camera 52, the ablation device 54 and/or the probe device 56 varies between 1 mm and 2 mm depending on the respective device and the respective type thereof. The outer diameter of the guide wire 24 is significantly smaller than the outer diameter of the elongated body 58 (see FIGS. 1b, 2b, 3b, 4b)

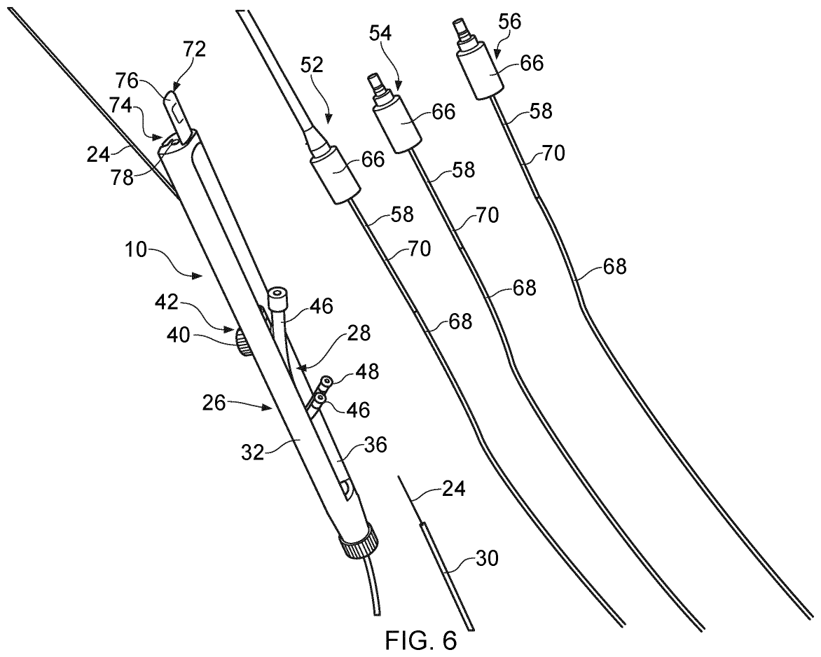

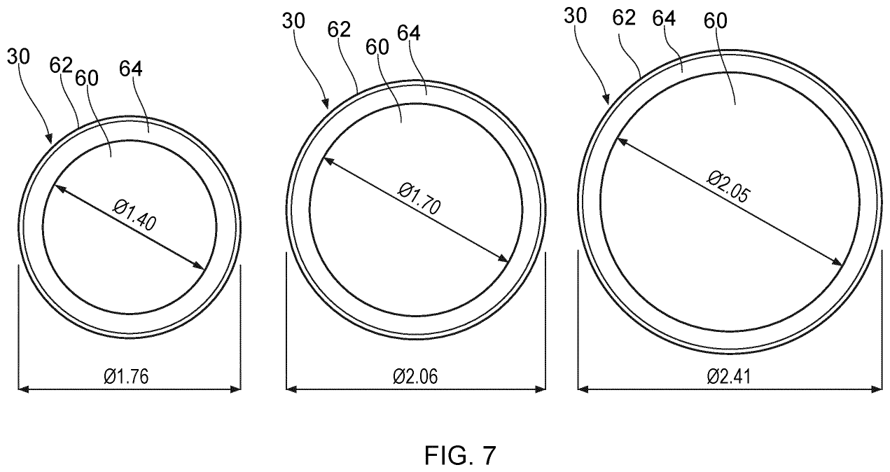

A catheter kit may include the guide wire 24, the catheter 10, for example as described above, and one or more of the camera 52, the ablation device 54 and the probe device 56 (see FIG. 6). The camera 52, the ablation device 54 and/or the probe device 56 may include a bulge 66 and the elongated body 58. The bulge 66 may be a connector, for example for connecting the ablation device 54 to a frequency generator and/or for connecting the probe device to an analysis device and/or a light source. In case of the camera 52, the bulge 66 may not be configured as a connector but as a part of the elongated body 58 of the camera 52 having an increased diameter. In particular, the elongated body 58 extends from the distal end to an analysis device and/or display device of the camera 52.

The bulge 66 may have an outer diameter that is greater than the outer diameter of the second passage 46. Thus, the bulge 66 may act as a stopper such that the elongated body 58 cannot further be inserted into the body 30. The length of the elongated body 58 between the distal end and the bulge 66 may be longer than the length of the body 30. The difference in the length of the elongated body 58 and the body 30 determines how far the elongated body 58 can be pushed out of the distal end of the body 30.

The elongated body 58 may include a first section 68 (visible in FIGS. 2b, 3b, and 4b) and a second section 70 which is arranged between the first section 68 and the bulge 66. The first section 68 preferably is flexible, while the second section 70 is rigid such that the second section 70 is less inclined to bend in comparison to the first section 68. The second section 70 may be made from a material different to the one of the first section 68. Alternatively, the first section 68 and the second section 70 are made from the same material while the second section 70 includes a sleeve made from a rigid material. The rigid sleeve may be incorporated into the second section 70 and/or arranged on its outer surface.

The rigidity of the second section 70 in conjunction with the rigidity of the insertion device 28 allows that the camera 52, the ablation device 54 and/or the probe device 56 protrude from the second passage 46 as depicted in FIGS. 2a, 3a and 4a. This means the camera 52, the ablation device 54 and/or the probe device 56 do not bend after exiting the second passage 46. This arrangement simplifies gripping the camera 52, the ablation device 54 and/or the probe device 56 since these devices 52, 54, 56 do not swing after exiting the second passage 46. What is more, the rigidity of the insertion device 28 and of the second section 70 provide support for holding the camera 52, the ablation device 54 and/or the probe device 56.

The second section 70 may have a visual appearance that differs from the visual appearance of the first section 68 such that a user can differentiate between the first section 68 and the second section 70. For example, the first section 68 and a second section 70 may have different colours and/or different surface characteristics. For example, the difference in the visual appearance may be constituted by the rigid sleeve. The length of the first section 68 may be chosen such that, when the first section 68 is completely inserted into the second passage 46 (i.e. it can be no longer seen), the distal end of the first section 68 coincides with the distal end of the body. The difference in the visual appearance of the first section 68 and the second section 70 may be used to position the distal end of the elongated body 58 at the distal end of the body 30.

The second section 70 may also include markers, labels and/or scales which indicate how far the elongated body 58 protrudes from the distal end of the body 30. The markers, labels and/or scales may be printed onto the second section 70 and/or can be constituted by one or more protrusions and/or recesses arranged on the outer surface of the second section 70.

The support device 26 may include a locking means 72 and an orientating device 74. The locking means 72 may allow to releasably fix the guide wire 24 with respect to the support device 26, for example by clamping the guide wire 24 to the support device 26. Optionally, the locking means 72 includes a flap 76 which is foldably attached to the support device 26, in particular to the support body 32. The flap 76 may include an elongated slit through which the guide wire 24 can be passed through. Depending on the position of the guide wire 24 in the elongated slit and on the orientation of the flap 76 with regard to the support device 26, the guide wire 24 can be clamped or released. However, other means for clamping and/or releasably fixing the guide wire 24 to the support device 26 are possible.

Similar to the rigid second section 70, the guide wire 24 can be supported after exiting the first passage 44 by the orientating device 74. The orientating device 74 may include a central opening 78 and/or a labyrinth shaped slit 80 (see FIG. 5). The central opening 78 may be oriented parallel to the longitudinal direction and, in particular, extends coaxial to the first passage 44. The central opening 78 may be arranged on a side of the channel 36 which opposes that side of the channel through which the body 30 enters the channel 36. Preferably, the central opening 78 is configured as a channel i.e. it extends in the longitudinal direction over a certain length such that the central opening 78 provides support to the guide wire 24 and, in particular, helps to maintain the orientation of the guide wire 24 parallel to the first passage 44.

The optional labyrinth shaped slit 80 allows insertion of the guide wire 24 to the central opening 78. In particular, the labyrinth shaped slit 80 provides a passage between the central opening 78 and the surroundings of the support device 26. Thus, the labyrinth shaped slit 80 allows insertion of the guide wire 24 while providing sufficient support that the guide wire 24 remains in the central opening 78.

The slit 38 may be positioned on that side surface of the support device 26 into which the labyrinth shaped slit 80 terminates, i.e. is open to the surrounding. This allows that the guide wire 24 is inserted into the first passage 44 through the slit 38 and then inserted into the central opening 78 by means of the labyrinth shaped slit 80.

A method of handling the catheter 10 and/the catheter kit is described in the following. The body 30 is inserted into the work channel of the insertion tube 16 via the biopsy port 18. When the distal end of the body 30 reaches the head 22 of the insertion tube 16, the support device 26 is attached to the biopsy port 18 of the scoping device 12. At this point, the slider 34 is positioned away from the biopsy port 18 as depicted in FIG. 1a.

Then, the guide wire 24 is inserted into the first passage 44 and advanced to the distal end of the body 30. At the same time, the guide wire 24 may be arranged within the central opening 78. Alternatively, the guide wire 24 is inserted into the body 30 (i.e. the first lumen 60) prior to the insertion of the body 30 into the insertion tube 16.

The guide wire 24 is then pushed out of the distal end of the body 30 and, thus, out of the head 22 as depicted in FIGS. 1b and 1c. The distal end of the guide wire 24 can then be positioned at the desired location by using the insertion tube 16. The position of the guide wire 24 in the longitudinal direction is locked or fixed by means of the locking means 72. Then, the body 30 is slit over the guide wire 24 by moving the slider 34 from its position as depicted in FIG. 1a to the position as depicted in FIGS. 2a, 3a, 4a and 6 i.e. from the position away from the biopsy port 18 towards the biopsy port 18. This sliding movement of the body 30 over the guide wire 24 is possible since the guide wire 24 is fixed with respect to the support body 32 while the slider 34 and, thus, the body 30 is moved with respect to the support body 32. As a consequence, the distal end of the guide wire 24 and a distal end of the body 30 are located at the same position.

After unlocking the guide wire 24, the guide wire 24 is then removed from the body such that the first lumen 60 is empty. However, the distal end of the body 30 remains in the desired location since the body 30 is not moved with respect to the guide wire 24.

Then, one of the camera 52, the ablation device 54 and the probe device 56 is inserted into the first lumen 60 via the second passage 46. These devices 52, 54, 56 are pushed into the body 30 until the distal end of the elongated body 58 is located at the same position at the distal end of the body 30. This can be checked by using the visual difference between the first section 68 and the second section 70. As the distal end of the body 30 is arranged at the desired position, the distal end of the camera 52, the ablation device 54 or the probe device 56 is also positioned at the desired location. Depending on the action to be performed, the distal end of the elongated body 58 may be pushed out of the distal end of the body 30 (as depicted in FIGS. 2a, 3a, and 4a) by pushing the elongated body 58 further into the second passage 56 or by retracting the body 30 by means of the slider 34 and simultaneously pushing the elongated body 58 into the second passage 56 by the same amount with which the body 30 is retracted. The latter means the distal end of the elongated body 58 remains at the desired location while the distal end of the body 30 is retracted away from the desired location.

The last step may be repeated by another one of the camera 52, the ablation device 54 and the probe device 56. In this way, the camera 52, the ablation device 54 and the probe device 56 can be used at the desired location one after the other. The arrangement of each one of the camera 52, the ablation device 54 and the probe device 56 in the first lumen 60 is depicted in FIGS. 2b, 3b, and 4b.

To irrigate the desired location, the camera 52, the ablation device 54 and the probe device 56 may be removed from the first lumen 60. Then, fluid is pumped to the desired location via the third passage 48 and then sucked from the desired location via the fourth passage 50. For example, the third passage 48 is connected to a fluid pump while the fourth passage 50 is connected to a suction source.

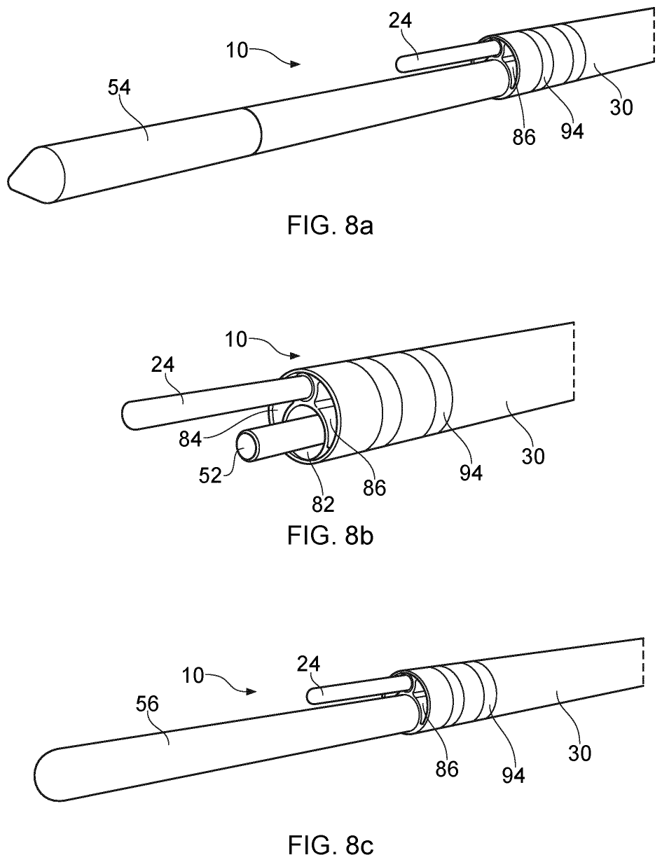

FIGS. 8 to 11 refer to a second embodiment. The second embodiment is identical to the first embodiment except the following differences. Thus, the description of the first embodiment equally applies to the second embodiment except for explicitly mentioned differences.

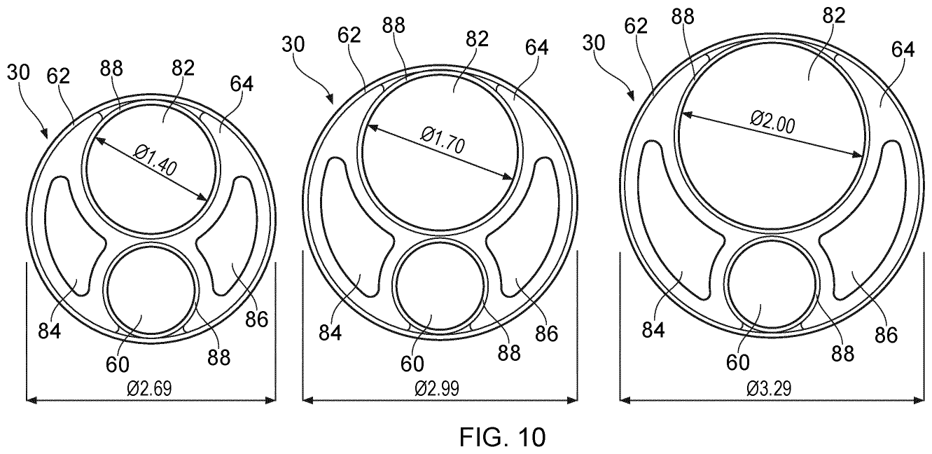

As visible in FIGS. 8a to 8c and 10, the body 30 includes a second lumen 82. Optionally, the body 30 further includes a third lumen 84 and a fourth lumen 86. In this embodiment, the first lumen 60 is in communication with the first passage 44 while the second lumen 82 is in communication with the second passage 46. Thus, as depicted in FIGS. 8a to 8c, 9 and 11b, the guide wire 24 and one of the camera 52, the ablation device 54 and the probe device 56 may be simultaneously arranged within the body 30. That is, the guide wire 24 is arranged within the first lumen 60 while one of the camera 52, the ablation device 54 and the probe device 56 is arranged in the second lumen 82.

The third lumen 84 may be in fluid communication with the third passage 48 while the fourth lumen 86 is in fluid communication with the fourth passage 50. The third lumen 84 and the fourth lumen 86 allowed to simultaneously pump fluid into the cavity within the body and suck the fluid out of the cavity while the guide wire 24 as well as one of the camera 52, the ablation device 54 and the probe device 56 is arranged in the cavity. This allows irrigation of the cavity while simultaneously operating one of the camera 52, the ablation device 54 and the probe device 56.

As depicted in FIG. 10, the first lumen 60, the second lumen 82, the third lumen 84 and/or the fourth lumen 86 may be defined by the outer shell 62 and the inner material structure 64 (see first lumen 60 and second lumen 82) and/or by the inner material structure 64 alone (see third lumen 84 and fourth lumen 86). The first lumen 60 and/or the second lumen 82 may be provided with a liner tube 88 which may contribute to the reduction of the friction between the body 30 and one of the guide wire 24, camera 52, the ablation device 54 and the probe device 56.

Examples of the diameters of the first lumen 60, the second lumen 82, the third lumen 84, the fourth lumen 86 and/or the body in millimetre can be gathered from FIG. 10.

Optionally, the liner tube 88 of the first lumen 60 may protrude from the proximal end of the body 30 and extends into the first passage 44. Similarly, the liner tube 88 of the second lumen 82 may protrude from the proximal end of the body 30 and may extend into the second passage 46. Such a configuration helps to provide the communication of the first passage 44 with the first lumen 60 and/or the second passage 46 with the second lumen 82. Additionally, the first lumen 60 and the second lumen 82 are sealed from the fluid in the third passage 48 and fourth passage 50 in the common passage 51 of the insertion device 28.

A method for handling the catheter 10 and/or the catheter kit according to the second embodiment is similar to the one of the first embodiment except for the following differences.

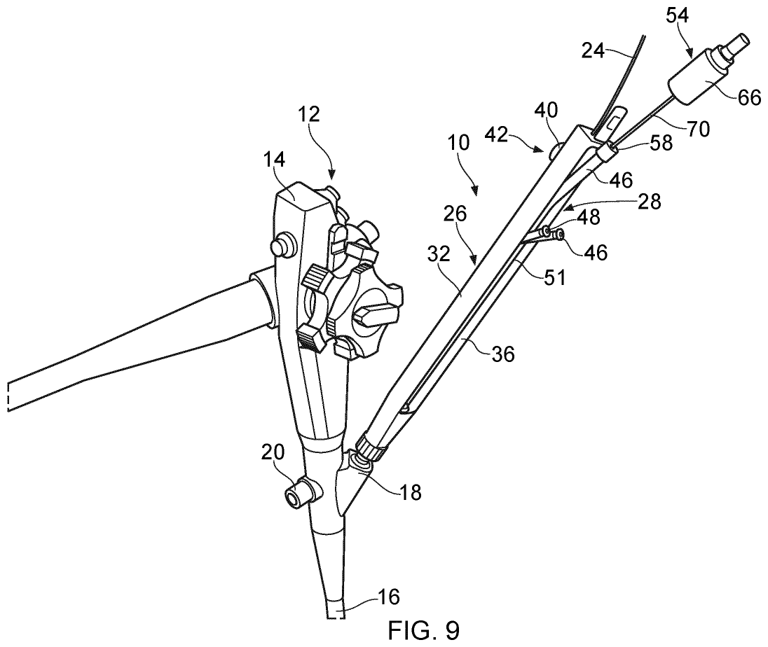

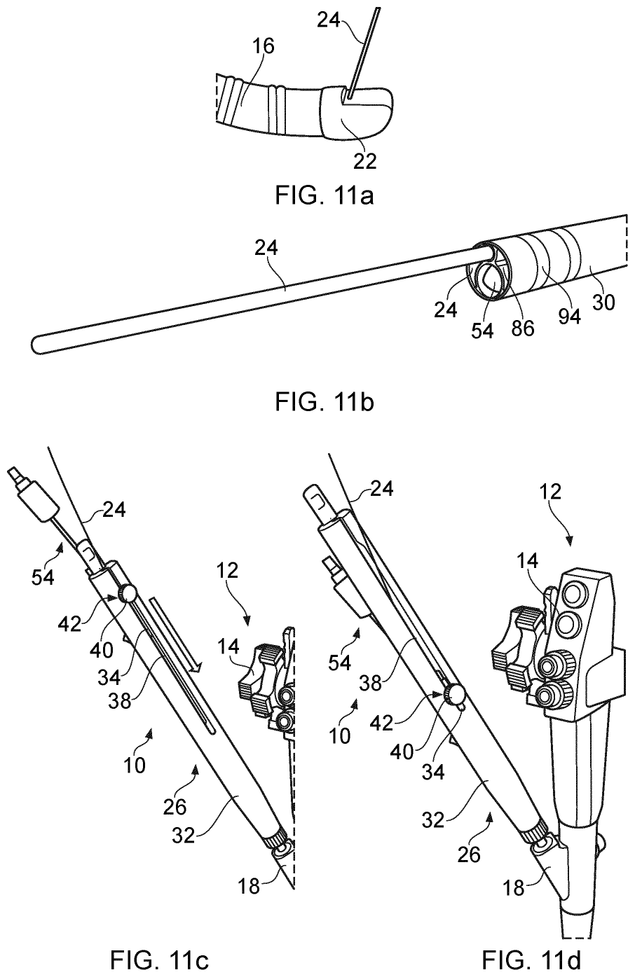

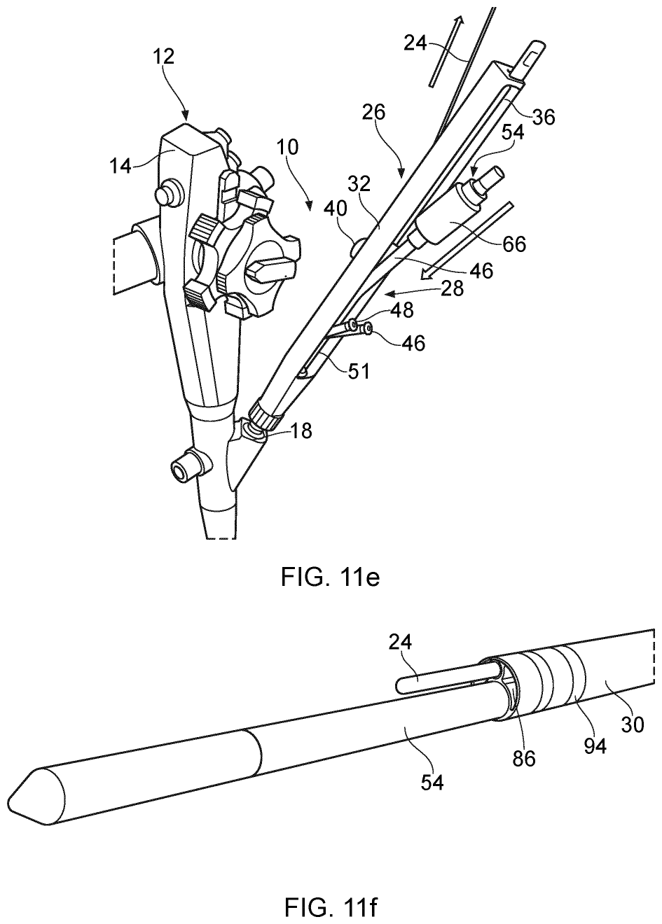

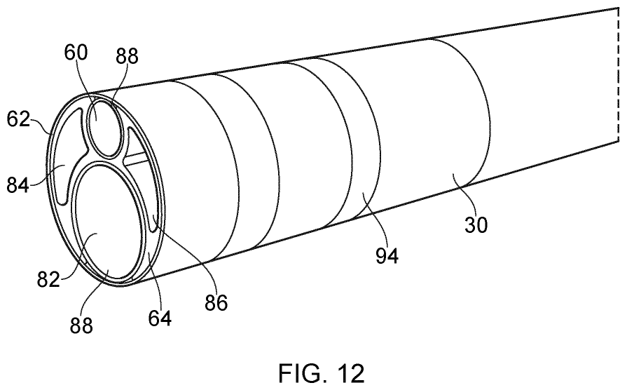

The guide wire 24 and one of the camera 52, the ablation device 54 and the probe device 56 can be positioned within the body 30 at the same time due to the provision of the first lumen 60 and the second lumen 82. In short, the guide wire 24 as well as one of the camera 52, the ablation device 54 and the probe device 56 are arranged within the body 30 such that their distal end coincides with the distal end of the body 30. At the same time, the slider 34 is arranged in a position away from the biopsy port as depicted in FIGS. 9 and 11c. Then, as depicted in FIGS. 11a and 11b, the guide wire 24 is pushed out of the distal end of the body 30 and is positioned at the desired location using the head 22. The guide wire 24 is then locked using the locking means 72.

As depicted in FIGS. 11c and 11d, the slider 34 is then pushed towards the biopsy port 18 such that the body 30 is slid over the guide wire 24. As one of the camera 52, the ablation device 54 and the probe device 56 is arranged within the body 30 and the body 30 is fixed to the insertion device 28, one of the camera 52, the ablation device 54 and the probe device 56 is simultaneously moved with the body 30 such that, by moving the slider 34, both the distal end of the body 30 and the distal end of the elongated body 58 of one of the camera 52, the ablation device 54 and the probe device 56 are positioned at the distal end of the guide wire 24 and, thus, at the desired location.

As depicted in FIG. 11e, the guide wire 24 is pulled back i.e. further into the first lumen 60, and the slider 34 is pushed towards the biopsy port 18 such that the distal end of the elongated body 58 of one of the camera 52, the ablation device 54 and the probe device 56 protrudes from the distal end of the body 30 (see FIG. 11f). At the same time, the slider 34 may be moved away from the biopsy port 18 such that the distal end of the body 30 is retracted a bit ending up in the situation as depicted in FIG. 11.

By removing one of the camera 52, the ablation device 54 and the probe device 56 of the body 30 and inserting another one of the camera 52, the ablation device 54 and the probe device 56 into the second lumen 82, these devices 52, 54, 56 may be employed at the desired location one after the other. The arrangement of each one of the camera 52, the ablation device 54 and the probe device 56 in the second lumen 82 is depicted in FIGS. 8a to 8c.

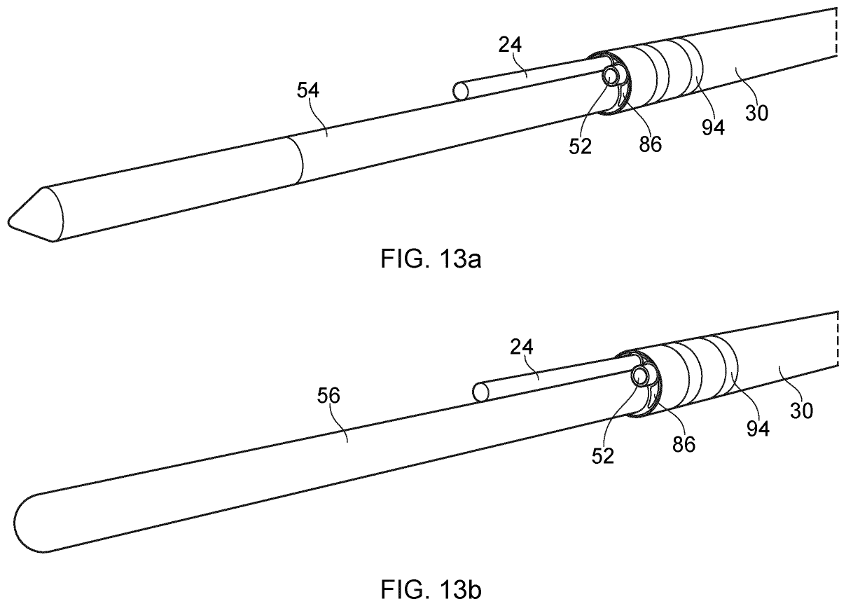

FIGS. 13 to 16 refer to a third embodiment. The third embodiment is identical to the first and/or the second embodiment except the following differences. Thus, the description of the first and/or second embodiment equally applies to the third embodiment except for explicitly mentioned differences.

The body 30 according to the third embodiment additionally includes a fifth lumen 90 (see FIGS. 13a, 13b and 15). The fifth lumen 90 is shaped to receive the elongated body 58 of the camera 52. The diameter of the elongated body 58 of the camera 52 can be smaller than the diameter of the elongated body 52 of the ablation device 54 and of the probe device 56. In particular, the diameter of the elongated body 58 of the ablation device 54 and the probe device 56 may be similar. Thus, the provision of the fifth lumen 90 allows to separately house the camera 52 while providing a lumen (in particular the second lumen 82) for receiving the elongated body 58 of the ablation device 54 or the probe device 56. Thus, it is possible to simultaneously operate the camera 52 and one of the ablation device 52 and the probe device 56.

The elongated body 58 of the camera 52 may be fixed within the fifth lumen 90. In this case, no clearance gap between the elongated body 58 of the camera 52 and the inner material structure 64 defining the fifth lumen 90 may be provided. Alternatively, the elongated body 58 of the camera 52 may be slidable within the fifth lumen 90 as described above in conjunction with the second lumen 82.

In the embodiment that the camera 52 is fixed to the body 30, the fifth lumen 90 may not be provided with a liner tube 88 as depicted in FIG. 15. The diameters of the body and the second lumen 82 in millimetre are also depicted in FIG. 15.

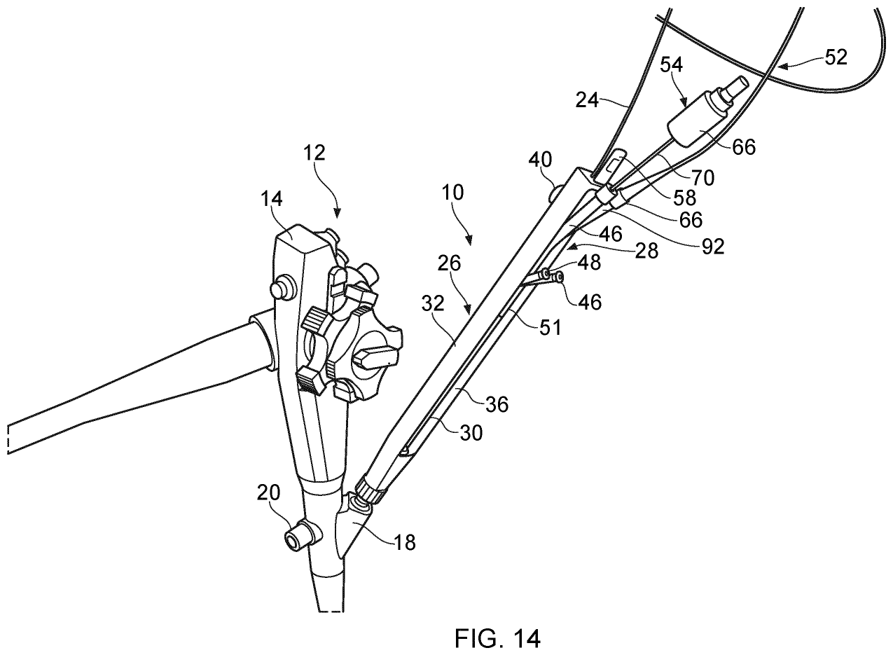

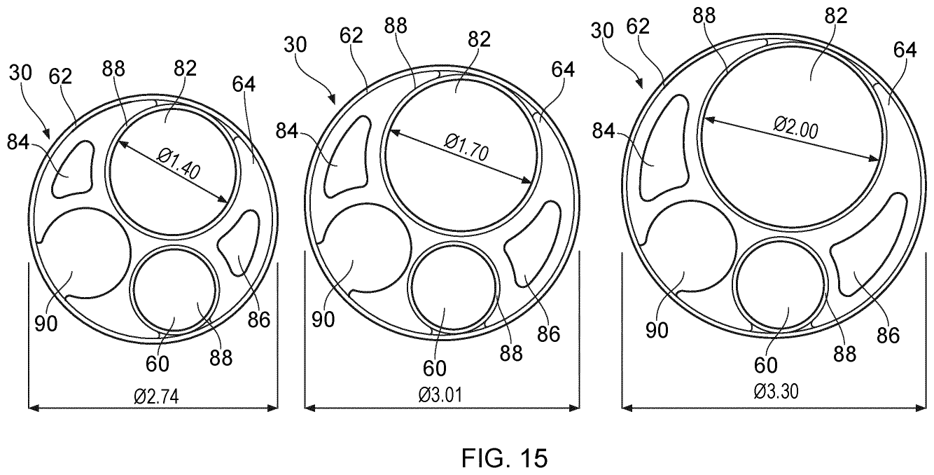

As depicted in FIG. 14, the insertion device 28 includes a fifth passage 92 for receiving the elongated body 58 of the camera 52. This allows to simultaneously insert one of the ablation device 52 and the probe device 56 on the one hand and the camera 52 on the other hand. The fifth passage 92 may be arranged at the same position in the longitudinal direction as the second passage 46. The fifth passage 92 may also be inclined to the common passage 51 and/or the first passage 44, preferably by the same angle as the second passage 46.

The body 30 may include a radio marker 94 at its distal end. In the embodiment shown, the radio marker 94 includes two metal rings arranged spaced apart from each other. The radio marker 94 may have an absorption of x-rays different to the surrounding tissue and/or other parts of the catheter 10 such that the distal end of the body 30 can be easily recognised in X-ray images or in computed tomography (CT) images. The radio marker 94 may have different shapes and configurations and/or may be provided with the other embodiments disclosed herein. In addition, although not depicted in the figures, the radio marker 94 may be provided with the catheter 10 according to the first and second embodiment.

A method for handling the catheter 10 and/or the catheter kit according to the third embodiment is similar to the one of the first and/or the second embodiments except for the following differences.

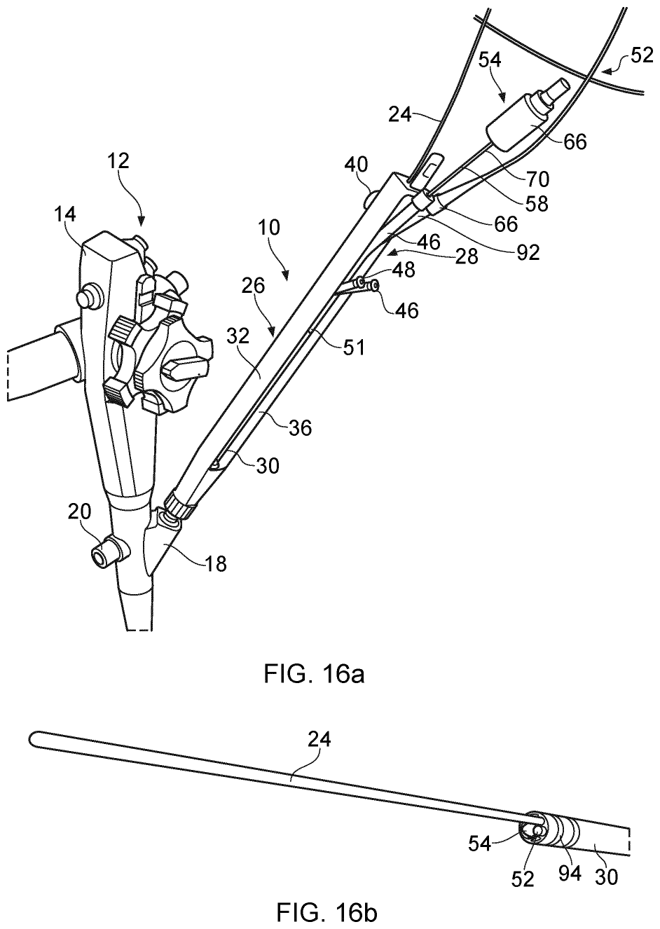

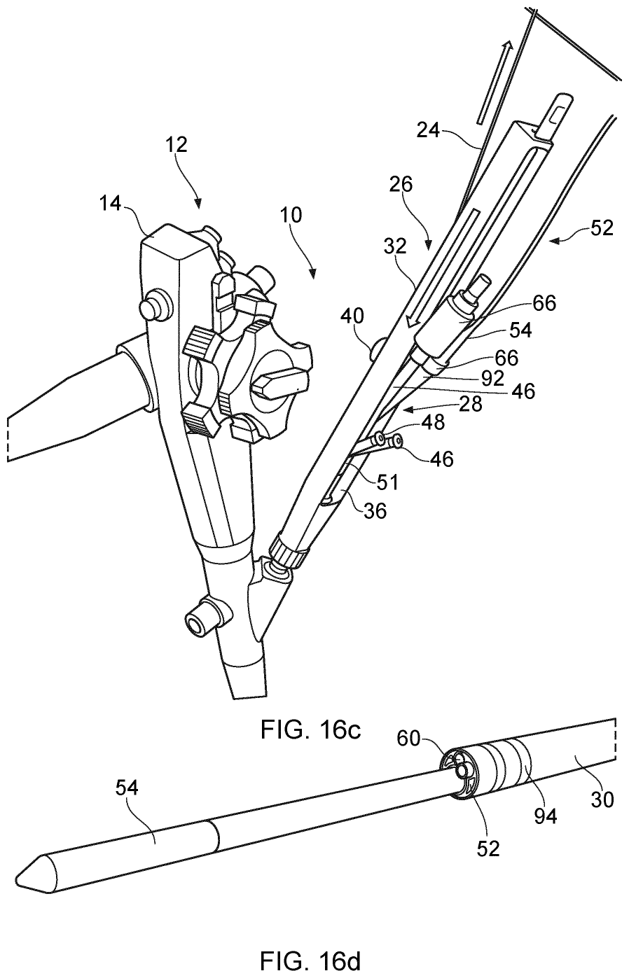

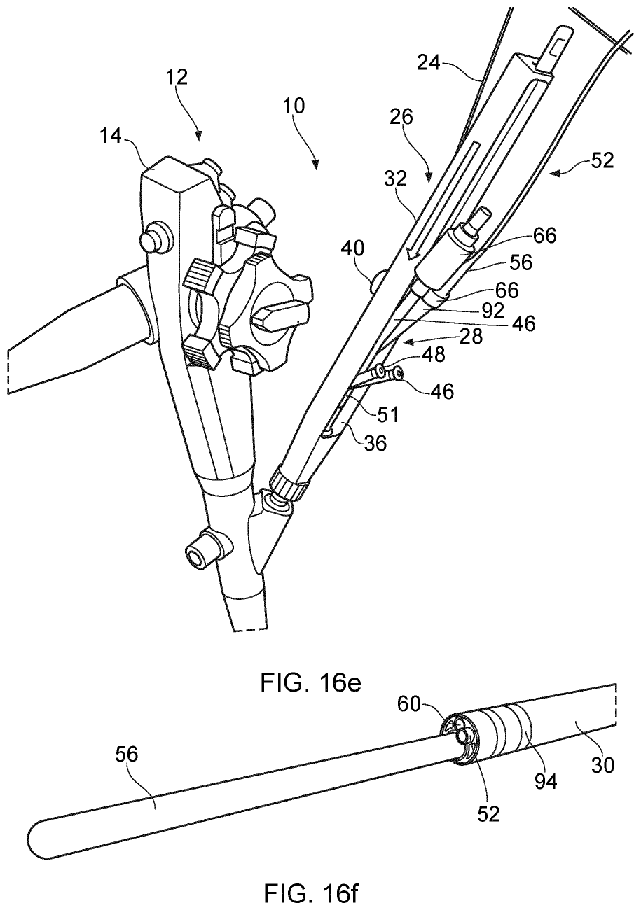

FIGS. 16a to 16f depict steps in the method for handling the catheter 10 and/or the catheter kit. At the beginning as depicted in FIG. 16a, the guide wire 24, the camera 52 and one of the ablation device 54 and the probe device 56 are inserted into the body such that their distal ends are located at the distal end of the body 30. As depicted in FIG. 16b, the guide wire 24 is pushed such that it distal end protrudes from the distal end of the body 30. At this point, the distal end of the guide wire 24 is positioned at the desired location. Then, the guide wire 24 is locked with regard to the support device 26 by means of the locking means 72.

As depicted in FIG. 16c, the insertion device 28 is moved towards the biopsy port 18 by sliding the slider 32 within the channel 36. As the guide wire 24 is fixed with respect to the support body 32, the body 30 along with the elongated bodies 58 of the camera 52 and one of the ablation device 52 and the probe device 56 are slid over the guide wire 24. This results in that the distal ends of all of the guide wire 24, the body 30, the camera 52 and one of the ablation device 52 and the probe device 56 are located at the same position, namely at this desired location. Then, one of the ablation device 52 and the probe device 56 may be pushed out of the distal end of the body 30 by moving the bulge 66 towards the second passage 46 i.e. by pushing the elongated body 58 of one of the ablation device 52 and the probe device 56 through the second lumen 82 (see FIG. 16d).

If the other one of the ablation device 54 and the probe device 56 is intended to be used at the desired location, the one of the ablation device 54 and the probe device 56 is removed from the second lumen 82 and the other one of the ablation device 54 and the probe device 56 is inserted into the second lumen 82 via the second passage 46 (see FIGS. 16e and 16f). The arrangement of the ablation device 54 or the probe device 56 in the second lumen 82 is depicted in FIGS. 13 and 13b.

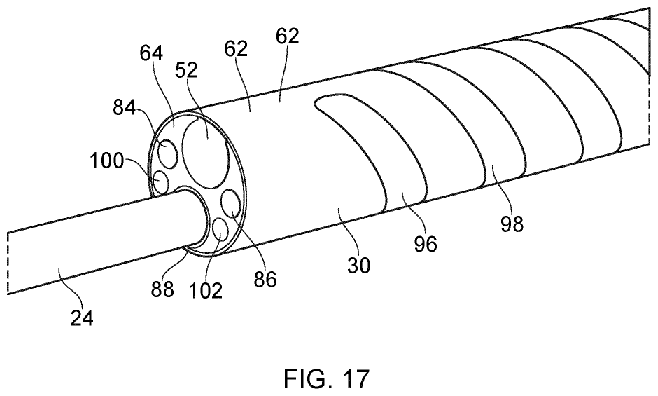

FIG. 17 refers to a fourth embodiment. The fourth embodiment is identical to the first to third embodiments except the following differences. Thus, the description of the first to third embodiments equally applies to the fourth embodiment except for explicitly mentioned differences.

The body 30 of this embodiment may include a first electrode 96 and/or a second electrode 98 which are arranged at the distal end of the body 30. The electrodes 96, 98 may be alternatively or additionally provided to the radio marker 94. In the depicted embodiment, the first electrode 96 and a second electrode 98 have this shape of a helix which extends around the outer shell 62 of the body 30. The two helices of the first electrode 96 and the second electrode 98, respectively, are intertwined such that, along an arbitrary line along the extension of the body 30, a section of the first electrode 96 is positioned between two sections of the second electrode 98 and vice versa.

The first electrode 96 and/or the second electrode 98 may protrude from the outer shell 62. It is also possible that the outer shell 62 includes a recess in which the first electrode 96 and/or the second electrode 98 are arranged. The outer shell 62 is preferably made from an electrically insulating material such that the first electrode 96 is electrically insulated from the second electrode 98.

The body 30 may further include a sixth lumen 100 and/or a seventh lumen 102 which extend from the proximal end to the distal end of the body 30. A first wire may be arranged within the sixth lumen 100 and/or a second wire may be arranged within the seventh lumen 102 (not visible in the figures). The first wire may be connected to the first electrode 96 and/or the second wire may be connected to the second electrode 98. The first wire and/or the second wire may extend beyond the proximal end of the body 30 to be connected to an electrical energy source such as a radio frequency generator. Thus, the first electrode 96 and the second electrode 98 may be provided for cutting tissue using a radio frequency electrical field between the electrodes 96 and 98. As indicated above, only one electrode and only one lumen for housing one wire may be provided with the body 30.

The distal end of the sixth lumen 100 and/or of the seventh lumen 102 may be closed at the distal end such that no fluid may enter into the sixth lumen 100 and/or the seventh lumen 102.

A method for handling the catheter 10 and/or the catheter kit according to the fourth embodiment is similar to the one of the first to third embodiments except tissue may be cut by means of the first electrode 96 and/or the second electrode 98.

1. A catheter configured to be inserted into a scoping device, comprising
a body having a distal end and a proximal end, and
an insertion device,
wherein the body includes at least a first lumen extending between the distal end and the proximal end, the first lumen being configured to receive at least one of a guide wire, a camera, an ablation device and a probe device,
wherein the insertion device is connected to the body at the proximal end and includes a first passage for receiving the guide wire and a second passage for receiving one of the camera, the ablation device and the probe device,
wherein the catheter further comprises a support device configured to be connected to a biopsy port of the scoping device,
wherein the support device includes a support body configured to be connected to the biopsy port and a slider slidable with respect to the support body,
wherein the proximal end of the body is fixed to the slider.

2. The catheter according to claim 1, wherein insertion device is rigid.

3. The catheter according to claim 1, wherein the insertion device further includes a third passage for introducing a fluid into the body and a fourth passage for emitting the fluid from the body.

4. The catheter according claim 1, wherein the support device can be rotatably connected to the biopsy port.

5. (canceled)

5. The catheter according to claim 1, wherein the support device further includes a locking device for releasably fixing the slider to the support body.

6. The catheter according to claim 1, wherein the support device includes a locking means for releasably locking the guide wire.

7. The catheter according to claim 1, wherein the support device further includes an orientating device for slidably supporting the guide wire in a longitudinal direction of the support device.

8. The catheter according to claim 1, wherein the first passage, the second passage, the third passage and/or the fourth passage are in fluid communication with the first lumen.

9. The catheter according to claim 1, wherein the body further includes a second lumen extending between the distal end and the proximal end, wherein the first passage is in communication with the first lumen and the second passage is in communication with the second lumen.

10. The catheter according to claim 3, wherein the body further includes a third lumen extending between the distal end and the proximal end and/or a fourth lumen extending between the distal end and the proximal end, wherein preferably the third passage is in fluid communication with the third lumen and/or the fourth passage is in fluid communication with the fourth lumen.

11. The catheter according to claim 1, wherein the body further includes a fifth lumen extending between the distal end and the proximal end, wherein preferably the insertion device includes a fifth passage, and wherein further preferably the fifth passage is in communication with the fifth lumen.

12. The catheter according to claim 11, wherein the fifth lumen is configured to receive the camera and the second lumen is configured to receive the ablation device or the probe device.

13. The catheter according to claim 1, further comprising a radio marker arranged at the distal end of the body.

14. The catheter according to claim 1, further comprising a first electrode and/or a second electrode located on an outer surface of the body at the distal end.

15. The catheter according to claim 14, wherein the body includes a sixth lumen extending between the distal end and the proximal end and a seventh lumen extending between the distal end and the proximal end, wherein a first wire is arranged in the sixth lumen and connected to the first electrode and/or a second wire is arranged in the seventh lumen and connected to the second electrode.

16. The catheter according to claim 14, wherein the first electrode and the second electrode have a spiral shape.

17. A catheter kit, comprising
a catheter,
a guide wire, and
one or more of the group including a camera, an ablation device and a probe device,
wherein the catheter includes a body having a distal end and a proximal end,
wherein the body includes at least a first lumen extending between the distal end and the proximal end, the first lumen being configured to receive at least one of the guide wire, the camera, the ablation device, and the probe device,
wherein the catheter further comprises a support device configured to be connected to a biopsy port of the scoping device,
wherein the support device includes a support body configured to be connected to the biopsy port and a slider slidable with respect to the support body, wherein the proximal end of the body is fixed to the slider.

18. The catheter kit according to claim 17, wherein the camera, the ablation device, and/or the probe device include a bulge and an elongated body, wherein preferably the elongated body includes a first section and a second section which can be visually differentiated from the first section and is positioned between the first section and the bulge, wherein further preferably a length of the first section is the same as a length of the body, and wherein further preferably the first section is more flexible than the second section.

19. A method for handling a catheter, wherein the catheter includes a body having a distal end and a proximal end, wherein the body includes at least a first lumen extending between the distal end and the proximal end,
wherein the catheter further comprises a support device configured to be connected to a biopsy port of a scoping device,
wherein the support device includes a support body configured to be connected to the biopsy port and a slider slidable with respect to the support body, wherein the proximal end of the body is fixed to the slider,
the method comprising the steps of
inserting a guide wire into the lumen of the body at the proximal end,
pushing the guide wire through the lumen until the guide wire protrudes from the distal end,
sliding the body over the guide wire until the distal end of the body is located at a distal end of the guide wire,
removing the guide wire from the lumen while the body is not moved,
inserting one of a camera, an ablation device and a probe device into the lumen of the body at the proximal end, and
pushing the one of the camera, the ablation device and the probe device through the lumen until a distal end of the one of the camera, the ablation device and the probe device protrudes from the distal end of the body.
